(19) United States
(12) Patent Application Publication
(10) Pub. No.: US 2020/0342550 A1
(43) Pub. Date: Oct. 29, 2020

(54) METHODS AND SYSTEMS FOR GENERATING RESTAURANT RECOMMENDATIONS

(71) Applicant: THE MEWAH CORPORATION, Alhambra, CA (US)

(72) Inventor: Michael C. Halimsaputera, Pleasant Hill, CA (US)

(21) Appl. No.: 16/855,400

(22) Filed: Apr. 22, 2020

Related U.S. Application Data
(60) Provisional application No. 62/837,558, filed on Apr. 23, 2019.

(51) Int. Cl. G06Q 50/12; G06F 16/9535; G16H 20/60; G06F 16/2457; G06Q 30/06; G06N 20/00

(52) U.S. Cl. CPC G06Q 50/12; G06F 16/9535; G06N 20/00; G06F 16/24578; G06Q 30/0631; G16H 20/60

(57) ABSTRACT

Examples are described for restaurant recommendation systems and methods for assisting a user or group of users with discovering, locating, selecting, patronizing, and reviewing restaurants that match dietary restrictions/preferences and/or other contextual information for the user and/or group of users. In one embodiment, a restaurant recommendation system includes a communication interface for communicating with a user device of a user, a processor, and a storage device storing instructions executable by the processor to; compare user information including dietary restrictions of the user to restaurant information including menu items for a plurality of restaurants, select a restaurant from the plurality of restaurants based on menu items of the restaurant matching the dietary restrictions of the user, and provide a recommendation of the restaurant to the user.

### CROSS-REFERENCE TO RELATED APPLICATIONS

The present application claims priority to U.S. Provisional Patent Application No. 62/837,558, entitled “METHODS AND SYSTEMS FOR GENERATING RESTAURANT RECOMMENDATIONS,” filed on Apr. 23, 2019. The entire contents of the above-identified application are hereby incorporated by reference for all purposes.

### FIELD

The disclosure relates to providing restaurant recommendations and interfacing users with restaurants.

### BACKGROUND

Mobile device applications frequently perform discrete tasks that require information from outside of the application. For example, many map applications will provide directions once you enter an address, but frequently you need to locate the address from a different source before you can use the map application. Review sites may provide ratings, but may not provide information as to hours of operation or menus for associated businesses. Looking at each restaurant website individually is time consuming and may not provide either ratings or relative locations.

Recent developments in food-related services have largely centered on food delivery options, taking advantage of the gig economy that has arisen around concepts such as ride-sharing, personal shopping, and other short-term, task-based jobs. However, current solutions only provide part of the information needed to make a decision regarding restaurant selection. For example, restaurant selection may be based on static criteria, such as average restaurant review scores that are relevant to a general population, but are not tailored to the particular user performing the restaurant selection. Further, given the increasing number of dietary restrictions many people have developed, there is currently no mechanism for sorting restaurants based on the requirements of a specific individual or group of individuals based on dietary restrictions. There is additionally no mechanism for coordinating preferences, locations, and dietary restrictions among groups of people. There is therefore an unmet need for an application that provides all of the desired information, presenting individuals with options that meet their individual preferences and dietary restrictions, and are continually updated to reflect changes in both restaurant offerings and personal preferences.

### SUMMARY

The services available to users for interacting with restaurants may only offer limited information regarding restaurant offerings and may not anticipate a user's preferences with regards to restaurant selection. For example, using conventional systems, a user may search multiple websites individually to find a particular restaurant of interest, search multiple different websites to read reviews of the restaurant, visit a website of the restaurant and/or call the restaurant to validate the availability of special dietary requirements of the user, and call, visit, or utilize a food ordering application and/or delivery service to order and pay for food. In order to rate the experience at the restaurant, the user may again manually navigate to a review website at a later time and write a review. The above manual steps may be complicated even further if the user is unsure of a particular restaurant of interest and/or if the user is part of a group of people, each of whom have different requests to be considered when selecting a particular restaurant of interest.

The disclosure provides systems and methods that learn preferences and dietary restrictions of users, build detailed restaurant menu and review databases through automated mechanisms that leverage artificial intelligence routines, and provide ratings and recommendations of restaurants to users based on comparing the learned preferences/dietary restrictions to the restaurant menu and review databases. In this way, a single user or a group of users may quickly identify restaurants of interest using a single resource that is tailored to the user(s) preferences and context.

In one example, a restaurant recommendation system includes: a communication interface for communicating with a user device of a user, a processor, and a storage device storing instructions executable by the processor. Instructions executable by the processor may compare user information including dietary restrictions of the user to restaurant information including menu items for a plurality of restaurants, select a restaurant from the plurality of restaurants based on menu items of the restaurant matching the dietary restrictions of the user, and provide a recommendation of the restaurant to the user. In this way, a user may be automatically notified of restaurants matching dietary restrictions of the user.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The disclosure may be better understood from reading the following description of non-limiting embodiments, with reference to the attached drawings, wherein below:

FIG. 1 shows a block diagram of an example restaurant recommendation system.

FIG. 2 shows a communication diagram of example interactions between components of a restaurant recommendation system.

FIG. 3 shows a flow chart of an example method for building and/or maintaining a menu for a restaurant menu database.

FIGS. 4A and 4B show a flow chart of an example method for generating a recommendation of a restaurant for one or more users and managing user interactions with the restaurant.

FIGS. 5-12 show example user interface screens for a restaurant recommendation application.

FIG. 13 shows an exemplary data structure for a restaurant menu database.

### DETAILED DESCRIPTION

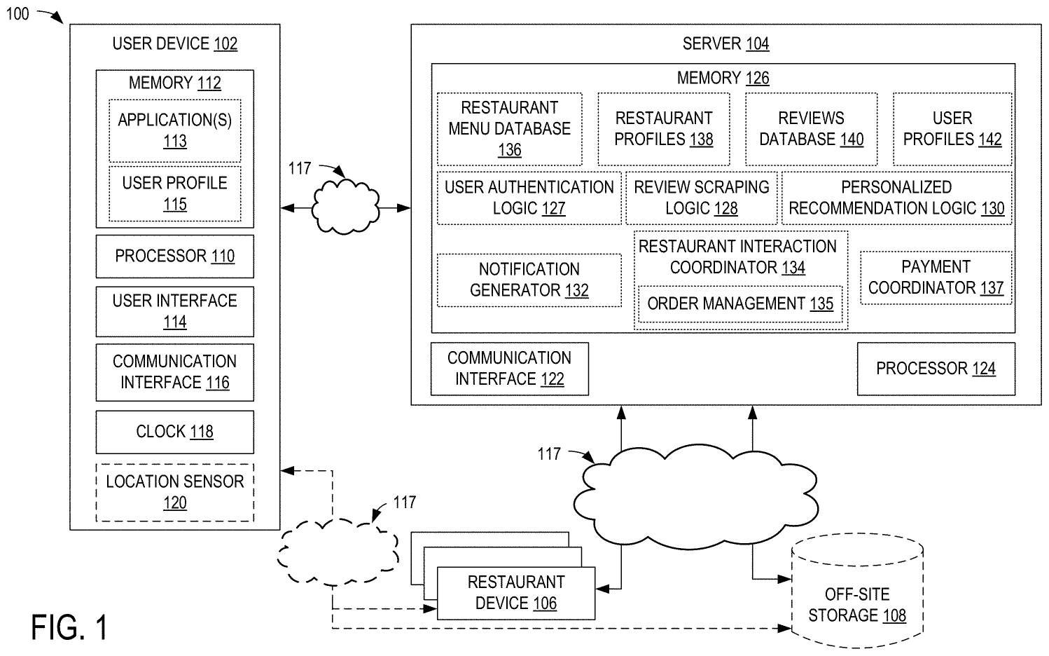

Examples are disclosed for assisting users in discovering, locating, selecting, and patronizing a restaurant that meets dynamic, real-time preferences of the users. FIG. 1 shows a block diagram of an example restaurant recommendation system 100 that may be used to provide a full-service restaurant experience, including learning a user's food-related preferences and restrictions, providing recommendations for food items and restaurants, connecting the user to the restaurant to place a food order for delivery/pick-up/dine-in, and collecting reviews from the user regarding the restaurant experience and food quality. The restaurant recommendation system 100 includes a user device 102, a server 104, one or more restaurant devices 106, and optionally an off-site (e.g., remote) storage device 108.

The user device 102 may include a mobile device such as a smartphone, tablet, wearable device, laptop, and/or another personal computing device. The user device 102 includes a processor 110 and memory 112 that stores data such as instructions executable by the processor to perform one or more of the operations described herein. For example, memory 112 may store application data to enable the user device 102 to run a restaurant recommendation application (e.g., to provide the user-side functionality of the restaurant recommendation system 100 described above). The application may control other components of the user device and/or retrieve information gathered by other components of the user device, such as user input received via a user interface 114, data from other devices received via a communication interface 116, time information received by a clock 118, and location information received by a location sensor 120. The application may also provide information to other components of the user device, such as information to be output to the user via the user interface 114 and/or information to be output to another device via the communication interface 116. The application may also access other information stored in memory 112, such as a user profile 115 and/or other stored user information. It is to be understood that memory 112 may store application data for a plurality of applications 113, as well as data for an operating system of the user device 102. Although shown as a single component, memory 112 may include a plurality of memory modules and/or storage devices, and may include volatile memory (e.g., RAM), non-volatile memory (e.g., hard disk drives, solid state drives, etc.), and/or a combination of volatile and non-volatile memory such as, for example, static random access memory (SRAM), dynamic random access memory (DRAM), flash memory, read-only memory (ROM), etc.

User interface 114 may include a hardware interface for one or more user interfacing components of the user device 102 and/or one or more user interfacing components in communication with the user device 102. For example, user interface 114 may transmit and/or receive data from one or more user interfacing components comprising user input/output devices, such as a display, speaker, touch sensor (which may be a standalone sensor or integrated with a display to form a touch screen), keyboard, mouse/pointing device, microphone, camera, biometric scanner, and/or other devices that interface with a user. Communication interface 116 may include a hardware interface for one or more communication components of the user device 102. For example, communication interface 116 may transmit and/or receive data from one or more external/remote devices (e.g., server 104, restaurant device(s) 106, off-site storage device 108, etc.) via one or more communication mechanisms, such as an antenna and/or data-carrying wire/cabling. The communication interface 116 may be configured for any suitable type(s) of communication, including wireless communication (e.g., Wi-Fi, BLUETOOTH, Near-Field Communication (NFC), cellular (e.g., 3G, 4G, 5G etc.), and/or other wireless mechanisms/protocols) and/or wired communication (e.g., Ethernet). The communication interface 116 may communicate with other devices directly and/or via one or more networks 117. Such networks 117 may be public/private or hybrid networks including, but not limited to, the Internet, a personal area network, a local area network (LAN), a wide area network (WAN) or a wireless local area network (WLAN) using any past, present, or future communication protocol (e.g., USB 2.0, USB 3.0, etc.).

Clock 118 and location sensor 120 represent example components for detecting or tracking a current context of a user and/or the user device 102 (e.g., a current day/time and a current location of the user/user device), however, it is to be understood that other components may be included in the user device 102 for such user/device context tracking. For example, the location sensor 120 may include a Global Positioning System (GPS) sensor and/or an inertial measurement device (e.g., accelerometer, gyroscope, etc.) for determining a heading direction/orientation, speed, and/or acceleration of the user/user device. The user device 102 may also include and/or be in communication with one or more sensors or data sources for determining weather/temperature around the user, a current, recent, and/or planned activity of the user (e.g., user schedule), a current mood of the user, contacts of a user, demographics of the user (e.g., age, economic status, race, gender, etc.), and/or other information that may be used to recommend a restaurant for the user. Some or all of the above information may be stored in the user profile 115 in memory 112 in some examples.

Although shown as a single computing device, server 104 may alternatively be formed of a collection of computing devices distributed on a network (e.g., a cloud-based server system) in some examples. Accordingly, the components of server 104 may be understood as being distributed in any suitable manner on one or a plurality of computing devices. Server 104 may include a communication interface 122, a processor 124, and memory 126. Communication interface 122 may include a hardware interface for one or more communication components of the server, in an analogous manner to the communication interface 116 of the user device 102. Accordingly, the description of the communication interface 116 also applies to the communication interface 122. Memory 126 stores data such as instructions executable by the processor 124 to perform one or more of the operations described herein. For example, memory 126 may store instructions for modules such as user authentication logic 127, review scraping logic 128, personalized recommendation logic 130, notification generator 132, restaurant interaction coordinator 134 (including order management module 135), and payment coordinator 137. Memory 126 may also store information that may be accessed by the above-described modules, such as a restaurant menu database 136, restaurant profiles 138, reviews database 140, and user profiles 142. As described above with respect to memory 112 of the user device 102, memory 126 is illustrated as a single component, however it is to be understood that memory 126 may alternatively comprise a plurality of memory modules and/or storage devices, and may include volatile memory, non-volatile memory, and/or a combination of volatile and non-volatile memory. Although described in the illustrated example as being included in memory 126, which is local to the server 104, at least some of the data in the databases, profiles, and/or modules described above may be stored in a remote storage device, such as off-site storage device 108 (which may include one or more storage devices in one or more locations remote from the server 104), in other examples.

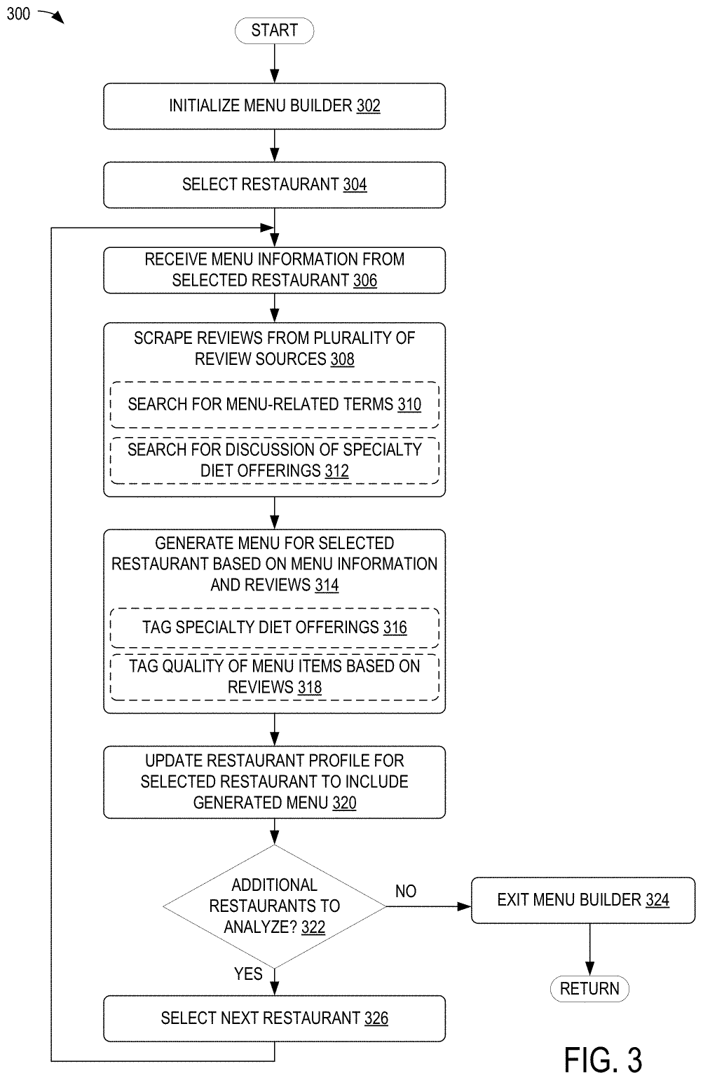

The restaurant menu database 136 may include a listing of menu items (e.g., food and/or drinks) for each of a plurality of restaurants. The listing of menu items for each restaurant may be generated and maintained via one or more mechanisms, including direct entry from the restaurant (e.g., communicated from an associated restaurant device 106 via communication interface 122), direct entry from a user (e.g., a regular user or a special administrative user) that visits or communicates with the restaurant directly (e.g., communicated from an associated user device 102 via communication interface 122), and/or automated entry from review scraping logic 128. In some aspects, the restaurant menu database 136 may also include ingredient lists for menu items. Such ingredient lists may be generic for a particular dish, or specific for the dish of a particular restaurant. Review scraping logic 128 may include instructions executable by the processor 124 to locate potential sources of menu item information (e.g., an official website for the restaurant, third-party review sites that host reviews for the restaurant, local reviews database 140, published articles that review the restaurant, social networking sites, etc.) and automatically analyze the reviews to identify references to particular menu items. Additional details regarding operation of the review scraping logic 128 are described below with respect to FIG. 3.

As the above-described menu item information sources may include additional details regarding specific menu items (e.g., quality, popularity, uniqueness/special features, etc.), such details may be included in the restaurant menu database for a given restaurant. Accordingly, for each restaurant in the restaurant menu database 136, a list of available menu items for that restaurant may be stored alongside tags or other notation protocols to indicate the associated details for the menu items (determined using the information gathering mechanisms described above). For example, each menu item for a restaurant may be tagged with one or more food category descriptors (e.g., a type of food/cuisine, a meal time associated with the menu item), one or more dietary descriptors (e.g., whether the food adheres to a dietary restriction, such as vegetarian, vegan, dairy-free, gluten-free, etc.), one or more ranking descriptors (e.g., whether the menu item is a specialty of the restaurant and/or a highest-rated/most often recommended menu item or menu item of a given food category for the restaurant), and/or other detailed information.

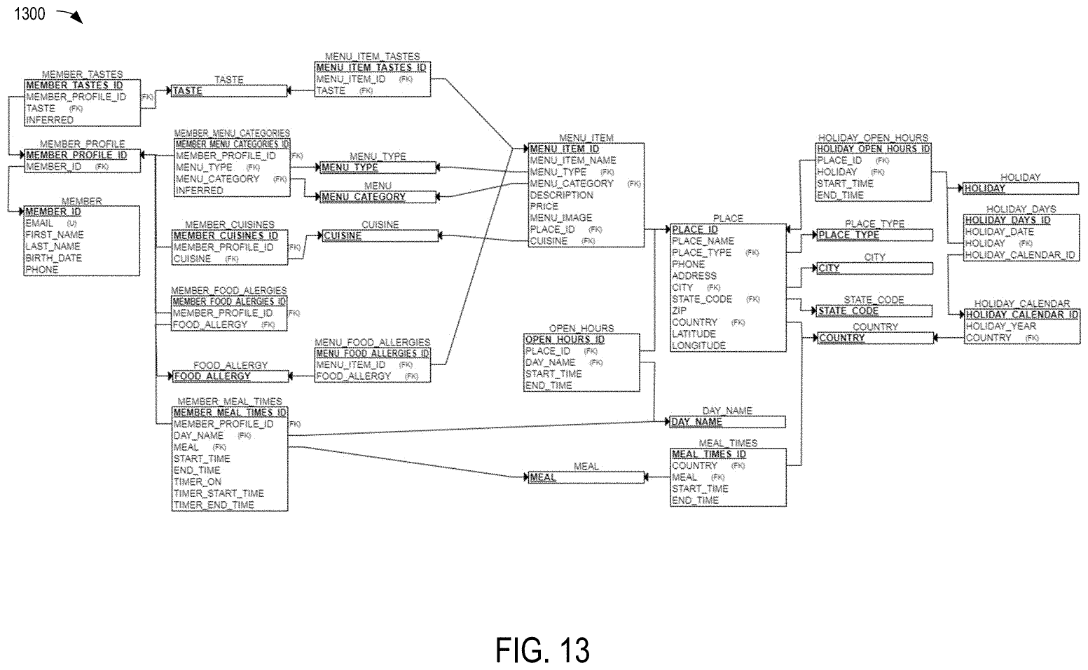

An example of a data structure 1300 for a restaurant menu database 136 is illustrated in FIG. 13. As shown, detailed information for a given restaurant may be stored in different containers or objects, which may be used to populate other containers or objects used in a restaurant menu database lookup procedure. For example, each menu item of the restaurant menu database may have attributes stored in a menu item container, including a menu item name, menu type, menu category, description, price, image, place identifier, and cuisine. Additional menu item information may be linked to the menu item container, including menu item taste, food allergy information, etc. The place identifier may point to a place container that provides detailed attributes for a restaurant associated with the menu item (e.g., place name, place type, phone, address, city, state code, zip code, country, latitude, longitude, etc.). Further restaurant information may be linked to the place container, including hours of operation, holiday designations, meal times, etc. The restaurant menu database may be accessed using user (e.g., member) information to find menu items that match dietary preferences/restrictions for a user and/or context information for the user. For example, containers including member profile information, such as member personal information (e.g., email address, phone number, name, birthdate, etc.), member tastes, favorite dishes, allergies, preferred cuisines/menu categories, meal times, etc. may be stored and/or accessed in order to find conforming menu items for the user. The user information may include inferred data (e.g., determined via artificial intelligence/machine learning algorithms as described herein) in some examples.

As described above, in some aspects the server 104 may locally maintain a reviews database 140, which includes reviews submitted by users of the restaurant recommendation system 100. For example, the server 104 may solicit reviews from users after determining that a user has had an interaction with a restaurant (e.g., dined at the restaurant or otherwise received food or service from the restaurant) and store the reviews in reviews database 140. Each review may be associated with a targeted restaurant (the subject of the review), and may include review information such as a time/date of the review, a time/date of the associated restaurant interaction, menu items ordered by the user, and/or other contextual information determined based on user interaction with the restaurant recommendation system 100.

The restaurant profiles 138 may aggregate (or point to) information for each of a plurality of restaurants from the restaurant menu database 136 and the reviews database 140, and store the aggregated information (or pointers) alongside general restaurant information for each respective restaurant, such as a restaurant location, restaurant cuisine, restaurant price range, restaurant contact information, etc. At least a portion of the information from the restaurant profiles 138 may be provided to a user if a user selects a restaurant (e.g., a recommended restaurant) via the application on the user device 102.

User profiles 142 may store (or point to) information for each of a plurality of users of the restaurant recommendation system 100. For example, one or more users of the user device 102 and/or other user devices may register with the server by providing login credentials (e.g., an email address, user name, password, etc.) to generate respective user profiles. The user profiles may then be populated with information regarding the respective user, which may be acquired through direct entry from the user, feedback from restaurants that interact with the user, and/or automatically determined via analysis of user interaction with the restaurant recommendation system 100 and/or other third-party systems (e.g., social networking sites, third-party review sites, etc.). The user profiles may include, for each user profiled therein, demographic information, dietary restrictions/preferences, historical activities/interactions, contact information, schedule information, and/or other information that may be helpful in determining restaurant recommendations for the user. Examples of dietary preferences may include detailed taste preferences such as preferred levels of sweetness, saltiness, spiciness, sourness, bitterness, etc. In some examples, the taste preferences may be correlated with other parameters, such as time of day, recent meals, recent activities, etc. For example, if a user is looking for an eating establishment after a typical dinner time (e.g., if the current time is in the late evening), the restaurant recommendation system may prioritize dessert and/or drinking establishments over other establishments due to the user's preference for eating dessert and/or having after-dinner beverages. Alternatively, if the user generally eats dinner in the late evening or other non-standard times, the system may prioritize establishments with meal service at atypical times. Such preferences may be determined based on user input and/or learned via artificial intelligence algorithms that examine historical eating habits of the user and/or of a group of users and determine correlations therebetween.

User authentication logic 127 may include instructions executable by the processor 124 to authenticate a user during a user login process. For example, when a user attempts to log in to a user account associated with the restaurant recommendation application executed on the user device 102, the user may enter login credentials, such as a user identifier and password (or the login credentials may be automatically generated, if the user previously saved the credentials to the user device). The login credentials may be transmitted to the server 104 (e.g., in a secured manner, such as via encrypted data transmission) and processed by the user authentication logic 127 in order to authenticate the user. For example, the user authentication logic 127 may confirm that the password matches the user identity entered/generated at the user device (e.g., by consulting a secured database, locally hosted by the server and/or hosted by a remote device, such as off-site storage device 108, that stores authentication information for users). The user authentication logic 127 may return a result of the confirmation to the user device 102. If the user authentication logic 127 confirms the login credentials, the user may be allowed to access the restaurant recommendation application associated with his/her user profile. Otherwise, the user authentication logic 127 may return an unauthorized result that causes the restaurant recommendation application to prompt the user for different login credentials, to prompt the user to create a new account and/or user profile, and/or to otherwise inform the user of the failed login attempt.

Personalized recommendation logic 130 may include instructions executable by the processor 124 to determine one or more restaurants that are predicted to be of interest to a user or group of users. The personalized recommendation logic 130 may collect contextual information from the user or group of users (including one or more of information from relevant user profiles 142, information from clock 118 indicating a time of day, transaction history (e.g., previous food orders placed via the server 104), information from location sensor 120 and/or other contextual sensors of the user device for each user, and filter restaurants from a first candidate pool to produce a second candidate pool based on the contextual information in the restaurant menu database 136, review data stored in review database 140, and/or restaurant profiles database 138. For example, time of day information may be used to target menu items that match a particular meal time (e.g., menu items tagged under the category “breakfast” may be preferentially recommended during a pre-determined window of time, such as from 6 AM to 11 AM). In another example, review data may be used to filter out restaurants from a first candidate pool to produce a second candidate pool, by removing restaurants with an average review rating below an average review rating threshold, wherein the average review rating threshold may be pre-selected by the user. In another example, dietary restrictions/preferences of each user may be used to filter out restaurants from the first candidate pool that do not have (or have less than a threshold number of) menu items that meet the dietary restrictions/preferences. In another example, location information (comprising user location and restaurant location) may be used to filter out restaurants that are more than a threshold distance (or more than a threshold travel time) away from a current location of a user, wherein travel time may be estimated based on GPS and traffic data available via the Internet.

Personalized recommendation logic 130 may be further configured to rank restaurants in the second candidate pool based on one or more factors, including an estimated travel time between locations of each restaurant in the second candidate pool and a location of the user (or group of users), a number of menu items of each restaurant in the second candidate pool matching the dietary restrictions of the user (or group of users), and an average review rating of each restaurant in the second candidate pool. In one example, personalized recommendation logic 130 may be configured to produce a ranked list of restaurants from restaurants in the second candidate pool, wherein, in one example, the ranked list comprises a first restaurant of the plurality of restaurants, wherein the first restaurant includes a first number of menu items matching the dietary restrictions of the user, and wherein the ranked list comprises a second restaurant of the plurality of restaurants, wherein the second restaurant includes a second number of menu items matching the dietary restrictions of the user, and wherein the first restaurant is ranked higher than the second restaurant in the ranked list in response to the first number of menu items exceeding the second number of menu items.

Personalized recommendation logic 130 may produce the ranked list by applying a mathematical formula that weights a plurality of scoring factors for each restaurant in the second candidate pool, and uses the weighted average score to determine a ranking of each restaurant, wherein, in one example, a restaurant with a greater weighted average score may be ranked higher than a restaurant with a lower weighted average score. In some examples, the weighting of each of the scoring factors may be determined via a machine learning algorithm, which may include one or more machine learning algorithms, including gradient descent. In another example, personalized recommendation logic 130 may select and/or rank restaurants based on output of a trained neural network, such as a deep neural network, wherein various features may be input into an input layer of the trained neural network, including one or more of restaurant and user location, user transaction history (e.g., previous food purchases/transaction conducted by the user), time of day, dietary preferences of the user, and menu items of the one or more restaurants, etc., and wherein the features may be mapped via the trained neural network to one or more inferred rankings/scores for the one or more restaurants.

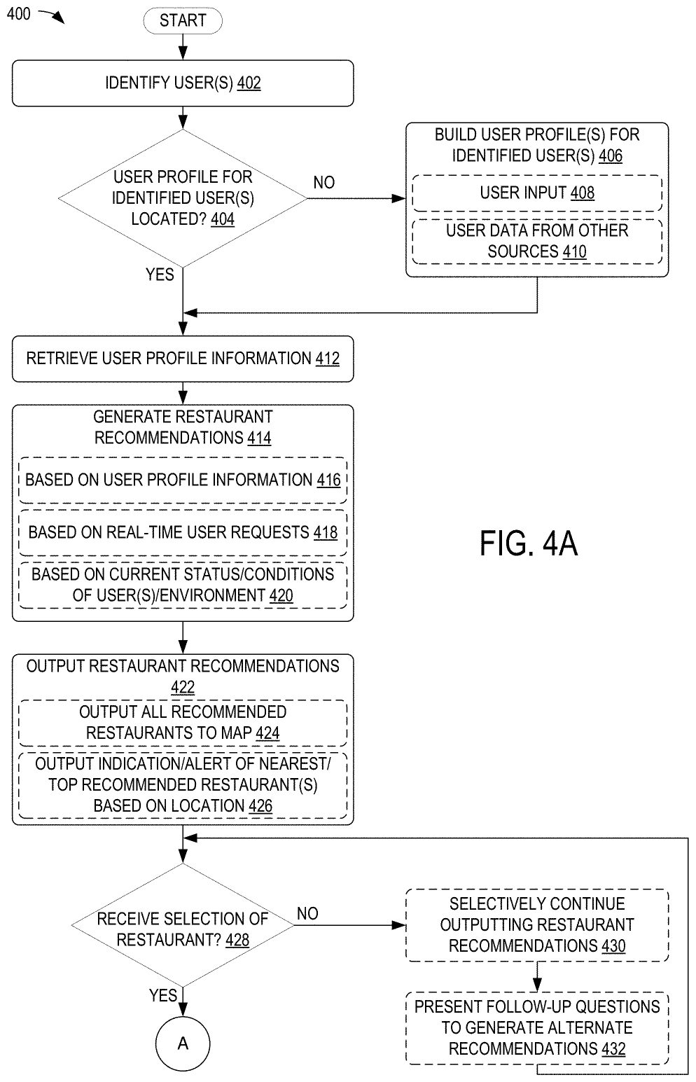

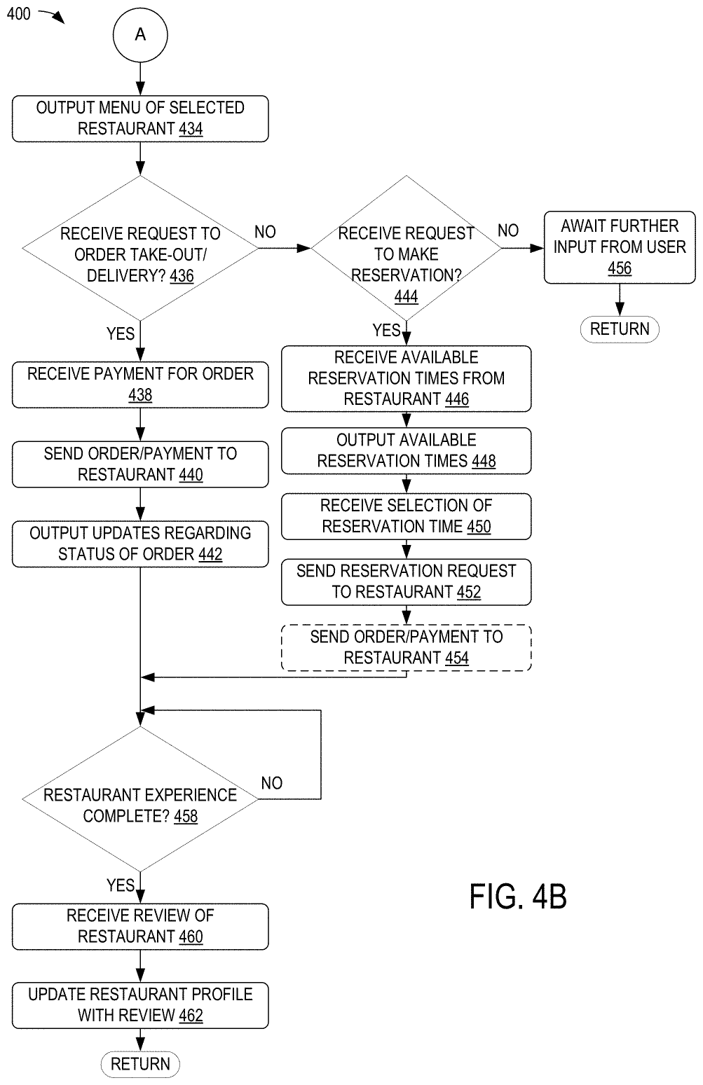

A hierarchy of the above factors may be dynamically inferred for each user based on the relative importance of each criterion to that user (e.g., determined via direct user input and/or via AI/machine learning). Based on the above filtering and ranking, higher/highest ranking restaurants may be selected that provide high ranking compliant menu items and are located near the user(s). More details regarding operation of the personalized recommendation logic 130 is described below with regard to FIGS. 4A and 4B.

Upon determination of one or more recommended restaurants, the recommendations may be presented via notifications coordinated by the notification generator 132. For example, the notification generator may generate instructions to be sent to the user device(s) of the user(s) to pop up a notification of at least a highest-ranking restaurant (as determined by personalized recommendation logic 130). In one example, the notification generator 132 may transmit notifications to a user device at a pre-determined time, wherein the pre-determined time may be selected by a user as part of a user account setup, or may be inferred via user transaction behavior (e.g., a most common time of day when a user makes purchases via the restaurant recommendation system 100). The notification may be presented on a map (e.g., with a marker showing a location of the highest-ranking recommended restaurant(s)) and/or as a standalone notification. The notification may be based on a push notification service that enables the server to send notification data to an associated application installed on a user device. The notification information may include custom text (e.g., SMS, MMS, etc.) alerts and/or dialogs that include images of menu items, descriptions of a restaurant and/or menu items (e.g., including information that led to the recommendation of the restaurant, such as location, menu items that match preferences of the user, etc.), and/or offers (e.g., coupons and/or deals that may be offered to a user, such as a discount that is only available for a limited period of time). In some examples, the notification generator may continuously send updated notifications as the user(s) changes location and/or rejects recommendations. For example, if a user rejects a recommendation for a highest-ranking restaurant and/or drives past the recommended restaurant, the notification generator may generate a follow-up recommendation of a next highest-ranking restaurant. The follow-up recommendation may utilize information from the rejection to reorganize the recommendations based on predicted reasoning for the rejection (e.g., restaurants offering different cuisine than a rejected recommended restaurant may be ranked higher than an original ranking responsive to the rejection).

Upon selection of a recommended (or other) restaurant (e.g., via the user device 102), the restaurant interaction coordinator 134 may be engaged to facilitate an ordering operation for the user(s), employing order management module 135 to handle order processing and fulfillment. For example, the restaurant interaction coordinator 134 may include instructions executable by the processor 124 to retrieve a menu for the selected restaurant from the restaurant menu database and output an indication of the menu items to the user device(s) 102 in order to allow the user(s) to select menu items and build an order. The restaurant interaction coordinator 134 (e.g., the order management module 135) may receive the order from the user device(s), calculate a cost associated with the order, and send a request for payment information to the user device(s). The restaurant interaction coordinator 134 may further transmit the order and/or payment information to the restaurant device 106 associated with the selected restaurant and/or to a third-party payment center. In some examples, the payment process may be handled by the payment coordinator 137. For example, the payment coordinator 137 may receive an indication of the cost of the order from the order management module 135 and coordinate communication between the user device 102, the server 104, and the restaurant device 106 (and/or a third-party payment processing device, such as a credit card and/or mobile wallet/payment processor) to send and process payment information for the user(s) to complete the order.

The restaurant interaction coordinator 134 may also receive from the user device(s) an indication as to whether the order is for dine-in, carry out, or delivery and provide this information to the restaurant device. The restaurant interaction coordinator 134 may maintain communication with the restaurant device 106 to provide updates on the submitted order (e.g., estimated time of availability), coordinate table reservations, and/or otherwise connect the user(s) to the restaurant as requested. Upon completion of an order (e.g., once the user has finished dining in, received the food via delivery, or picked up the food from the restaurant), the restaurant interaction coordinator 134 may request feedback from the user(s) regarding the restaurant experience and update the restaurant menu database (e.g., with feedback regarding the particular menu items that were ordered) and/or add a review for the selected restaurant in the reviews database 140 based on the user feedback.

Restaurant device 106 may include a server, laptop, and/or other computing device, configured to host restaurant data including a restaurant menu. In some examples, restaurant device 106 may comprise a cloud server configured to maintain a publically accessible restaurant associated website. Restaurant device 106 may comprise a processor, and memory that stores data such as menu items, restaurant location, restaurant hours of operation, menu item prices, menu item ingredients, etc. Restaurant device 106 may be communicatively coupled to one or more remotely located computing devices, including server 104 and user device 102, via a wired or wireless communication network. Although shown as a single component, restaurant device 106 may include a plurality of components, which may be remotely located, such as memory modules and/or storage devices, including volatile memory (e.g., RAM), non-volatile memory (e.g., hard disk drives, solid state drives, etc.), and/or a combination of volatile and non-volatile memory such as, for example, static random access memory (SRAM), dynamic random access memory (DRAM), flash memory, read-only memory (ROM).

Offsite-storage 108 may include a server or other computing device, configured to host data. Data hosted via offsite-storage 108 may comprise a structured repository of data, such as a database. In one example, offsite-storage 108 may store a database of user account information, including usernames, passwords, user preferences, user transaction histories, etc. Offsite-storage 108 may be configured to receive and process data requests submitted via remotely located computing devices, such as server 104. In some examples, offsite-storage 108 may comprise a cloud database configured to maintain a secure record of user account information for user accounts of a restaurant recommendation system. Offsite-storage 108 may comprise a processor, and memory, wherein the memory may comprise volatile memory (e.g., RAM), non-volatile memory (e.g., hard disk drives, solid state drives, etc.), and/or a combination of volatile and non-volatile memory such as, for example, static random access memory (SRAM), dynamic random access memory (DRAM), flash memory, read-only memory (ROM). Offsite-storage 108 may be communicatively coupled to one or more remotely located computing devices, including user device 102, server 104, and restaurant device 106, via a wired or wireless communication network. Although shown as a single component, offsite-storage 108 may include a plurality of components, which may be remotely located, such as memory modules and/or storage devices.

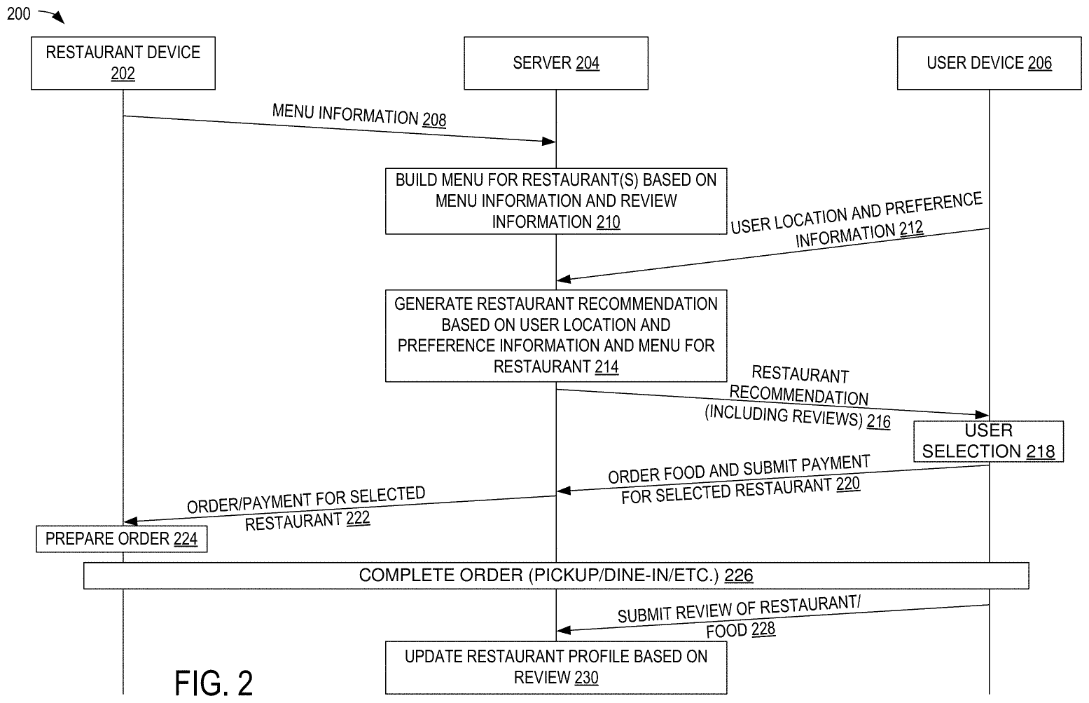

The above-described operation of the server components is further described in FIG. 2 as a communication diagram 200 illustrating example communications between components of a system (such as restaurant recommendation system 100 of FIG. 1) to provide an improved dining experience for a user that reduces manual operations that a user may perform relative to attempting to select a restaurant using different systems and resources. Communication diagram 200 illustrates example communications between a restaurant device 202, a server 204, and a user device 206. Restaurant device 202, server 204, and user device 206 may be examples of restaurant device 106, server 104, and user device 102 of FIG. 1, respectively, thus the associated description of these components in FIG. 1 likewise apply to the associated components of FIG. 2.

At 208, the restaurant device 202 sends menu information to the server 204. The menu information may include a listing of menu items and further information regarding each of the menu items, such as pricing information, dietary information (e.g., identifying specialty diets to which the menu item conforms), popularity (e.g., whether the menu item is something in which the restaurant specializes), etc. At 210, the server 204 builds a menu for one or more restaurants (including the restaurant associated with restaurant device 202) based on the received menu information and review information. For example, the server 204 may perform a review scraping operation to identify available menu items and user opinions on the menu items using review scraping logic, as described in more detail with respect to review scraping logic 128 of FIG. 1 and method 300 of FIG. 3.

At 212, the user device 206 sends user location and preference information. The preference information may include user input preferences for the user and/or preferences that are learned for the user (e.g., based on information received from historical user activities/actions/transactions, social networking activity of the user, user profiles, etc.). The preferences may include a type of food that the user is currently seeking, general food preferences for the user, dietary restrictions for the user, a distance/time to eating that the user prefers (e.g., whether the user is looking for a restaurant to order from now or in the future), etc. Although described with reference to a single user, it is to be understood that the user location and preference information may include information for multiple users seeking a group restaurant experience, where preferences for each of the users of the group is received at the server and used to provide a recommendation. Additionally, although not shown, it is to be understood that information such as user authentication information (discussed in more detail above with respect to user authentication logic 127 of FIG. 1) may be sent between the user device 206 and the server 204 in order to authenticate the user(s) and/or to determine which user profile is to be accessed for restaurant recommendation prior to sending the user location and preference information. Furthermore, the transmission of user location and preference information at 212 may be different for subsequent iterations of the communications. For example, the user location and preference information may include all user preference information in a first iteration (e.g., just after setting up a user account) in order to update a user profile for the user at the server 204. In a subsequent iteration, only user location and any updated preference information may be sent to the server 204 (e.g., to update the user profile).

At 214, the server 204 generates a restaurant recommendation (which may identify at least a highest-ranking restaurant) based on the user location and preference information (including any preference information stored at the server 204) and the menu for the restaurants (e.g., as built at 210). The restaurant recommendation may be determined using personalized recommendation logic 130 of FIG. 1 and/or method 400 of FIGS. 4A and 4B, as described in more detail below. At 216, the server 204 sends the restaurant recommendation (including information from the reviews of the associated restaurants) to the user device 206 for presentation to the user. For example, identifiers and location information for the restaurants may be provided to the user device for populating a map with markers corresponding to the recommended restaurants. Further information such as contact information, menu information, and reviews of the recommended restaurants may also be sent to the user device 206 to allow the user to view more information about a particular recommended restaurant (e.g., upon selection to view details of the particular recommended restaurant). The restaurant recommendation sent at 216 may include multiple recommendations provided over time (e.g., updated as a user changes location and/or makes selections/rejections of recommendations). For example, the restaurant recommendation may include push notification alerts (e.g., timer and/or location-based push notification alerts) transmitted from the server to the user device identifying a recommended restaurant for a user's current context and based on the user's profile (e.g., storing user preferences/restrictions, etc.). As a non-limiting, illustrative example, the timer for controlling the push notification alerts may be configured to present notifications at 30 minutes before a specified start meal time for a user (e.g., based on a user profile). In the example, if the user does not order a meal, the push notification alerts may be configured to repeat every 10 minutes, with updates based on any change in location of the user (e.g., if the user is in a different location, the repeated alerts may include suggestions of different restaurants that are closer to the user than the prior suggested restaurants). In the example, the timer for the timer-based notification alerts may end when the user selects a restaurant and/or completes a meal order and/or after 30 minutes past the specified end meal times for the user (e.g., based on the user profile).

At 218, a user of the user device 206 provides a selection of a restaurant from the recommendation provided at 216 and a selection of food/drink items to be ordered from the selected restaurant. At 220, the user device 206 sends the order and associated payment information to the server 204. The server 204 then propagates the order to the restaurant device 202 associated with the selected restaurant, as indicated at 222, and propagates payment information to a payment service associated with the restaurant. The order may include selection of items from the menu as built at 210 and/or special requests regarding modifications to the menu items and/or requests for items that are not on the menu. As noted above, in subsequent uses of the restaurant recommendation system, some transmitted information may be changed and/or omitted in light of prior uses of the system. For example, in a first use of the restaurant recommendation system, the user device may transmit payment information to the server to complete an order. However, the payment information may be stored at the server (e.g., in association with the user profile of the user, and responsive to a confirmation from the user accepting the storage of this information) after transmission of the payment information. In a subsequent use of the restaurant recommendation system, the user device may transmit to the server a request to use the stored payment information (e.g., responsive to the user selecting such an option for payment) instead of transmitting the actual payment information to the server.

At 224, the restaurant associated with the restaurant device 202 (e.g., the selected restaurant) prepares the order. At 226, the order is completed (e.g., the order is picked up or delivered or the user finishes a dine-in experience), and the user device 206 submits a review of the selected restaurant and/or food (from the order submitted at 220) to the server 204 at 228. In some examples, the review may be solicited by the server 204 after a threshold time has passed from the completion of the order at 226. In other examples, the user may proactively submit the review. The review may be submitted using the same application that was used to select the recommended restaurant and submit the order to the selected restaurant. At 230, the server 204 updates the restaurant profile and/or associated databases based on the review (e.g., to reflect the user's experience and/or ranking of information such as speed/quality/accuracy of service provided by the restaurant, quality of food ordered, other information regarding offerings by the restaurant, etc.). Accordingly, the user's review submitted at 228 may adjust how the selected restaurant is recommended to users in the future. For example, a high rating for a particular menu item may cause the selected restaurant to appear in a higher ranking of a recommendation for restaurants that serve the particular menu item than prior to the submission of the review.

FIG. 3 is a flow chart of an example method 300 for building and/or maintaining menus for one or more restaurants. Method 300 may be performed by a server of a restaurant recommendation system, such as server 104 of FIG. 1 (e.g., review scraping logic 128 of FIG. 1). At 302, the method includes initializing a menu builder. For example, an automated review scraping application may be initialized at set times (e.g., at a set frequency, such as once a day) and/or responsive to a trigger such as a receipt of additional restaurant information at the server. At 304, the method includes selecting a restaurant to be analyzed. At 306, the method includes receiving menu information from the selected restaurant. For example, menu information may include an indication of menu items served by the restaurant and other details regarding the menu items, as described above with respect to the menu information sent at 208 of FIG. 2 and the restaurant menu database 136 of FIG. 1.

At 308, the method includes scraping reviews from a plurality of review sources. The scraping of reviews may include searching for menu-related terms, as indicated at 310, and/or searching for discussion of specialty diet offerings, as indicated at 312. The scraping may pair particular menu-related terms with quality indicators associated with the menu-related terms in the reviews (e.g., if a food item term, such as a “burger,” is located near a quality rating term, such as “great,” the scraper may associate the burger from the restaurant as being of high quality). The scraping may also associate quality indicators with general restaurant parameters, such as ambience, service, menu variety, specialty diet compliance, etc. The scraping logic may utilize artificial intelligence (AI) and/or machine learning algorithms to parse the reviews and identify key terms for use in building a restaurant menu and profile. For example, the AI/machine learning may be used as a tone analyzer to determine emotions and/or tones in the reviews. Based on the determined tone of the review (e.g., whether the review brings joy and/or otherwise shows a positive emotion or whether the review brings anger and/or otherwise shows a negative emotion), the menu items mentioned in the review may be identified and added (when associated with positive emotions) or removed (when associated with negative emotions) from the restaurant menu recommendations.

The plurality of review sources accessed by the review scraping logic may include third-party review aggregators, social media sites, locally-hosted/maintained review databases, newspaper or other journalism-related websites, and/or other sources. In some examples, only a subset of reviews are scraped for a given restaurant from each source (e.g., a top five most recent and/or highest ranked reviews from each of one or more of the sources) in order to increase a speed of the scraping.

At 314, the method includes generating a menu for the selected restaurant based on the menu information (e.g., received at 306) and reviews (e.g., scraped at 308). In the generated menu, menu items may be tagged as specialty diet offerings, as indicated at 316, and/or with quality indicators (e.g., based on the reviews scraped at 308), as indicated at 318. In some examples, reviews from particular sources and/or authors and/or reviews that utilize particular terms may be weighted differently (e.g., higher) than other reviews when identifying information to be included in the generated menu. For example, during review scraping, three reviews for a particular menu item at a restaurant may be identified as attributing the quality indicator of “great” to the menu item, while one review may attribute the quality indicator of “bad” to the menu item. If the review that provided the “bad” quality indicator is from a highly-regarded site, the quality indicator associated with the food item in the generated menu may be skewed more toward “bad” than “great,” despite the greater number of reviews using the “great” quality indicator, due to the weighting of the review using the “bad” quality indicator. In other examples, equal weight may be provided to each review, such that a final quality indicator used in the generated menu is an average of the quality indicators found in the scraped reviews. In still other examples, the generated menu may compile all of the quality indicators and provide a summary of the total quality indicators from all of the scraped reviews. The full information from the reviews may be stored such that a user may access more information regarding the determination of the quality indicator in the generated menu.

At 320, the method includes updating a restaurant profile for the selected restaurant to include the generated menu and any associated data regarding the menu items of the generated menu (e.g., quality indicators, specialty diet compliance, etc.). At 322, the method includes determining if there are additional restaurants to analyze. If there are no more restaurants to analyze at the time (e.g., “NO” at 322), the method proceeds to exit the menu builder, as indicated at 324. If there is at least one additional restaurant to analyze (e.g., “YES” at 322), the method proceeds to select a next restaurant, as indicated at 326, and returns to 306 to receive menu information for the newly-selected restaurant. The remaining operations of the method are then performed for the newly-selected restaurant to build the associated menu for the newly-selected restaurant. Method 300 may be repeated for each new restaurant, and operations from method 300 may be performed periodically to maintain an updated menu for each restaurant (e.g., on a timed interval and/or responsive to a trigger such as an indication of new information for a restaurant).

The restaurant menu building described above with respect to FIG. 3 may be utilized to provide a restaurant recommendation for one or more users. An example of such a recommendation is shown in method 400 of FIGS. 4A and 4B. Method 400 may be performed by a server of a restaurant recommendation system, such as server 104 of FIG. 1 (e.g., personalized recommendation logic 130 of FIG. 1).

Starting on FIG. 4A, at 402, the method includes identifying one or more users. For example, method 400 may be used to provide a restaurant recommendation for a single user or a group of users taking into consideration a context, preferences, and/or other information for each of the users. Each user may be identified based on login credentials of the user (e.g., as the users log in to a restaurant recommendation application) and/or detected identifiers of the user (e.g., biometrics, user profiles associated with a user device that is connecting with the server, etc.). At 404, the method includes determining if a user profile for the identified user(s) is located (e.g., present on the server and/or accessible by the server). The user profile may refer to a user profile associated with the restaurant recommendation system and/or an associated application for using the restaurant recommendation system. If a user profile is not located for at least one identified user (e.g., “NO” at 404), the method proceeds to 406 to build a respective user profile for each user that does not already have an associated user profile. The user profile(s) may be generated directly based on information from user input (e.g., via responses to guided questions), as indicated at 408, and/or automatically generated based on information including user data from other sources, as indicated at 410. The user profile may include account information, such as an email address/contact information, user name, password, application settings, etc., as well as user preference information, such as dietary preferences/restrictions, location preferences, dietary habits, favorite food/drink items and restaurants, etc. The user profile may also include user contextual information and historical activities of a user, which may relate to interactions of the user with the restaurant recommendation system (e.g., prior restaurants visited by the user, reviews left by the user, etc.) and/or general actions of the user (e.g., user schedule, visited locations of the user, user reactions to various environments/conditions, etc.). The user profile may also include information regarding contacts of the user and associated preferences of the contacts.

If a user profile is located for each identified user (e.g., “YES” at 404) and/or once a user profile is generated for each identified user (e.g., after building the user profile(s) at 406), the method proceeds to 412 to retrieve user profile information. At 414, the method includes generating restaurant recommendations for the identified user(s). The restaurant recommendations may be based on user profile information, as indicated at 416 (e.g., the user profile information retrieved at 412 for each of the identified users), real-time user requests, as indicated at 418, and/or current status/conditions of the user(s)/environment, as indicated at 420. In a group scenario, the restaurant recommendations may be based on dietary restrictions and/or preferences, as well as the contextual information described above with respect to 416, 418, and 420, for each member of the group. For example, the server may compare the preferences and current context of the user(s) to information for each restaurant known to the server in order to determine a closest match(es) to the preferences and context of the user(s).

In some examples, information from the user profile and current context of the user may be weighted differently in determining restaurant recommendations. For example, a restaurant may be filtered out (e.g., not recommended in an initial recommendation) even though the restaurant matches several user preferences and is recommended by a contact of the user if the restaurant also is located outside of a threshold distance from the user. As another example, a restaurant that serves a highly-rated specialty food item that complies with a user's specialty diet restriction may be recommended at a higher ranking than a restaurant that serves more highly-rated food items in total. In such an example, compliance with the specialty diet of the user may rank more highly than the number of highly-rated food items (e.g., the variety of highly-rated food items) in determining a restaurant recommendation for the user. In a group scenario, the dietary preferences/restrictions and/or other factors of different group members may be weighed differently. For example, a parent's preferences may be weighed higher than a child's preferences, or a member with a greatest number of dietary restrictions or preferences may be weighed higher than the preferences of the other members of the group. The weighting of different preferences and other criteria for determining the recommendations may be automatically generated and/or adjusted based on user input specifying the importance of different preferences/criteria. In some examples of a group recommendation scenario, recommendations may be generated for each member of the group, then the overlapping restaurants (e.g., the restaurants recommended for all or a majority of users of the group) may be provided as recommended restaurants for the group. In other examples, one or more group members may be weighted higher than one or more other group members, and the recommendations of the more highly-weighted group members may be preferentially provided in the restaurant recommendations.

At 422, the method includes outputting restaurant recommendations (e.g., transmitting an indication of the recommended restaurants to one or more user devices associated with the identified user(s)). In one example, the restaurant recommendations may include a ranked list of restaurants, wherein the ranking of the restaurants in the ranked list is based on one or more of the above discussed factors. In an example, the ranked list comprises a first restaurant of the plurality of restaurants and a second restaurant of the plurality of restaurants, wherein a first ranking of the first restaurant is higher than a second ranking of the second restaurant in response to a first estimated travel time between the first restaurant and a user location being less than a second estimated travel time between the second restaurant and the user location.

Outputting the restaurant recommendations may include outputting information usable by a user device to display all recommended restaurants on a map, as indicated at 424, and/or to present an indication and/or alert of nearest/top recommended restaurants based on location information for the identified user(s), as indicated at 426. In some examples, a subset of the set of restaurants recommended at 414 may be output for presentation at the user device at 422, where the subset is selected based on a location of the user (e.g., the closest restaurants to the user), and where the subset is continuously updated to display the closest restaurants in the set of restaurants recommended at 414 as the user moves through an environment.

At 428, the method includes determining if a selection of a restaurant (e.g., one of the recommended restaurants or another restaurant) is received. If no selection is received (e.g., “NO” at 428), the method proceeds to 430 to optionally selectively continue outputting restaurant recommendations (e.g., maintaining an output of the same recommended restaurants and/or updating the output to reflect newly-recommended options). The method may further optionally include presenting follow-up questions to generate targeted alternate recommendations, as indicated at 432. The method then returns to check if a selection of a restaurant is received at 428.

If a selection of a restaurant is received (e.g., “YES” at 428), the method proceeds to output a menu of the selected restaurant, as indicated at 434 in FIG. 4B. For example, the menu may include the menu of the selected restaurant, as generated and maintained using a mechanism such as method 300 of FIG. 3. The method may then include determining if a request to order take-out or delivery is received, as indicated at 436. If a request to order take out or delivery is received (e.g., “YES” at 436), the method proceeds to receive payment for the order, as indicated at 438 and send the order and payment to the restaurant, as indicated at 440. The method may also include outputting updates regarding the status of the order, as indicated at 442.

If a request to order take-out or delivery is not received (e.g., “NO” at 436), the method proceeds to 444 to determine if a request to make a reservation at the selected restaurant is received. If a request for a reservation is received (e.g., “YES” at 444), the method proceeds to request and receive available reservation times from the selected restaurant, as indicated at 446, and output the available reservation times, as indicated at 448. At 450, the method includes receiving a selection of a reservation time, and a reservation request including the selected reservation time is sent at 452. The method further optionally includes sending the order and/or payment information to the restaurant ahead of the reservation time at 454, so that the restaurant may begin preparing the order for the user's arrival. In this way, the order may be prepared in light of the reservation timing to reduce wait time for the food (e.g., timed to be prepared at or within a short time period of when the user(s) arrives for the reservation).

If a request to make a reservation is not received (e.g., “NO” at 444), the method proceeds to 456 to await further input from the user. If no further input is received, the method may return to present recommended restaurants, or exit until further input from the user is received. If further input from the user is received, the method may proceed to an appropriate operation (e.g., to 438 if a request for a take-out/delivery order is received, to 446 if a request to make a reservation is received, to 434 if a different restaurant is selected, etc.).

At 458, the method includes determining if a restaurant experience is complete (e.g., if a user has received a take-out/delivery order and/or has completed a dine-in experience). If the restaurant experience is not complete (e.g., “NO” at 458), the method returns to continue waiting for completion of the experience. If the restaurant experience is complete (e.g., “YES” at 458), the method proceeds to receive a review of the restaurant from the user(s), as indicated at 460. The reviews may be received responsive to a solicitation by the server and/or instigated by the user(s). At 462, the method includes updating the restaurant profile with the received review.

In some embodiments, machine readable storage media may have executable instructions that, when executed, cause one or more processors to perform an operation comprising all or part the methods of FIGS. 3 and 4. Such machine readable storage may include any of a variety of storage media, such as magnetic storage media (e.g., magnetic tapes or magnetic disks), optical storage media (e.g. optical discs), electronic storage media (e.g. conventional hard disk drives, solid-state disk drives, or flash-memory based storage media or any other tangible storage media or non-transitory storage media.

In some embodiments, an apparatus such as one or more of the apparatus of FIG. 1 may comprise means for performing the various actions and/or operations of the methods of FIGS. 3 and 4.

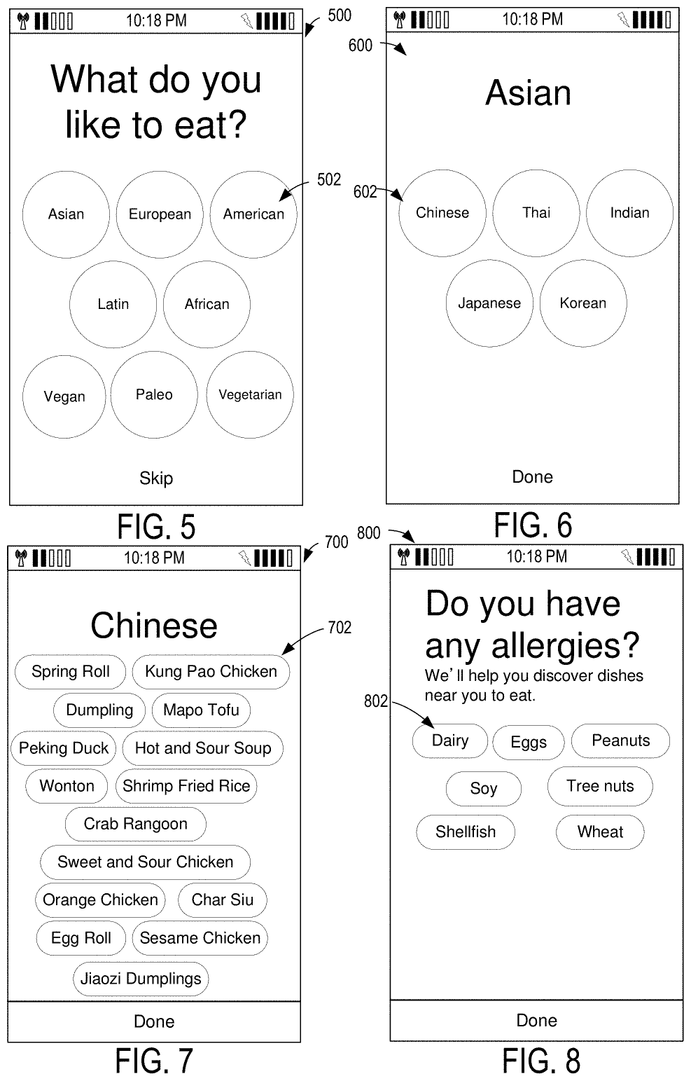

FIGS. 5-12 show example user interface screens for a restaurant recommendation application. For example, the user interface screens of FIGS. 5-12 may be presented on a user device, such as user device 102 of FIG. 1, running an application for interfacing with a restaurant recommendation system, such as restaurant recommendation system 100 of FIG. 1. FIGS. 5-8 show example user interface screens that may be used during a user profile setup process and/or during a request for restaurant recommendations. FIG. 5 shows a user interface screen 500 that prompts a user to indicate at least one regional cuisine in which the user is interested. Responsive to selection of one of the regional cuisine or dietary preference user interface elements 502, a screen showing more detailed cuisines associated with the selected regional cuisine may be displayed. For example, responsive to selecting the “Asian” cuisine user interface element on user interface screen 500, user interface screen 600 of FIG. 6 may be presented, providing user interface elements 602 that each represent a different particular region of Asian cuisine.

Responsive to selection of a detailed cuisine via one of the user interface elements 602, a screen showing popular food items associated with the selected cuisine may be presented. For example, responsive to selecting the “Chinese” cuisine user interface element on user interface screen 600, user interface screen 700 of FIG. 7 may be presented, providing user interface elements 702 that each represent a different food item associated with Chinese cuisine. One or more user interface elements 702 may be selected to indicate a preferred food item for the user, which may be stored in an associated user profile. The preferred food item may be indicated to be a general food preference, or may represent a current food preference (e.g., a food item that the user would like to consume at a present time) used to guide a current restaurant recommendation (e.g., targeting a search for a restaurant that provides a high quality offering of the selected food item(s) based on review scraping, as described with respect to FIGS. 3-4A, and/or other information).

FIG. 8 shows a user interface screen 800 that guides a user toward setting up a dietary preference and/or restriction indicator for an associated user profile. For example, the user interface screen 800 includes a plurality of user interface elements 802 that are selectable to indicate an allergy or aversion to the associated element. Responsive to a selection of any allergies, the restaurant recommendation system may tailor any restaurant recommendations to include only restaurants (or prioritize restaurants) that offer food items that comply with the dietary restrictions of the user. Restaurants that are known to provide high quality food items that meet dietary restrictions of the user (e.g., based on review scraping, as described with respect to FIGS. 3-4A, and/or other information) may also be prioritized in any recommendations provided to the user.

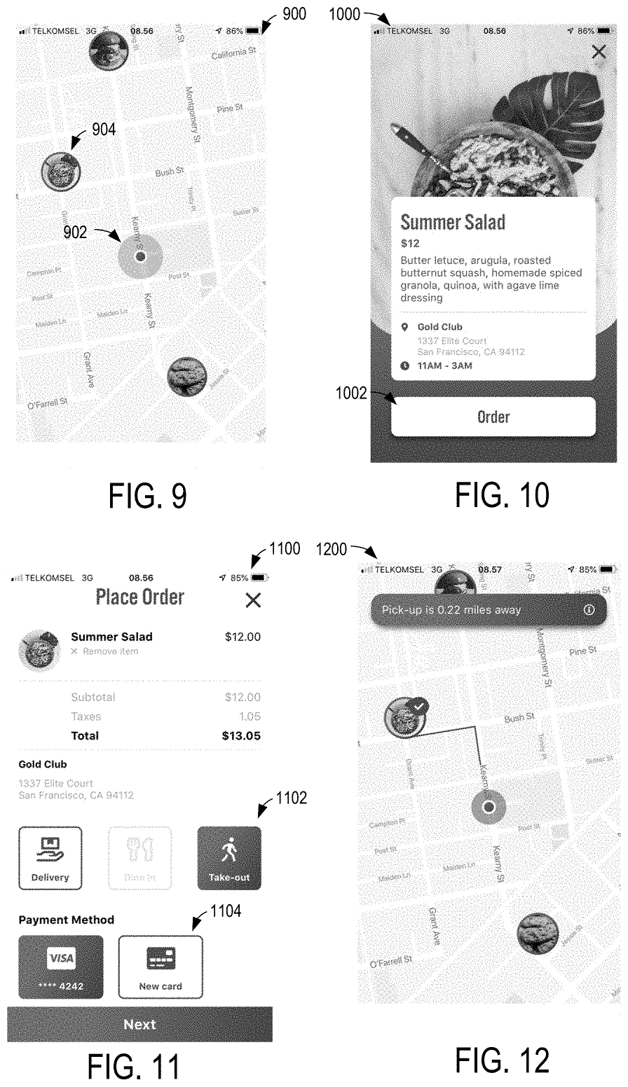

FIGS. 9-12 show example user interface screens for selecting a restaurant and placing a delivery order. For example, FIG. 9 shows a user interface screen 900, which displays a map with a current location marker 902 and a plurality of recommended restaurant markers 904. The recommended restaurant markers 904 may be automatically updated as the user moves through the environment in order to show nearby restaurants that meet user preferences. Responsive to selecting one of the recommended restaurant markers 904, user interface 1000 of FIG. 10 may be displayed, identifying an associated restaurant and a food item that is recommended based on the user's preferences and context. The user interface 1000 may include a user interface element 1002 that is selectable to place an order for the recommended item. For example, responsive to selecting user interface element 1002, user interface 1100 of FIG. 11 may be presented, showing a cost of the order and providing user interface elements 1102 to present options for receiving the order (e.g., delivery, dine-in, and take-out) and user interface elements 1104 to present options for paying for the order (e.g., using an previously-entered credit card or entering a new card to be used to pay for the order). Responsive to selecting take-out and a valid payment method via user interface screen 1100, user interface screen 1200 of FIG. 12 may be presented, which displays a distance and/or directions to the selected restaurant. In this way, the user may be guided to pick up the order.

The above-described examples simplify the restaurant selection and interaction process for users by providing a unified interface for the whole restaurant experience, including identifying a food item that the user may be interested in consuming, recommending a restaurant for acquiring food item(s) (including providing reviews and/or information regarding quality of food items at recommended restaurants), coordinating recommendations for a group of users, enabling users to place and pay for an order at a selected restaurant, guiding a user to receiving the food via delivery, dine-in, or take-out, and soliciting reviews of the selected restaurant. The above processes provide a dynamic approach to restaurant recommendations that automatically accounts for changing tastes in users and varying user contexts in order to provide targeted recommendations in a user-friendly manner.

The description of embodiments has been presented for purposes of illustration and description. Suitable modifications and variations to the embodiments may be performed in light of the above description or may be acquired from practicing the methods. For example, unless otherwise noted, one or more of the described methods may be performed by a suitable device and/or combination of devices, such as the server 104 and/or user device 102 described with reference to FIG. 1. The methods may be performed by executing stored instructions on machine readable storage media with one or more logic devices (e.g., processors) in combination with one or more additional hardware elements, such as storage devices, memory, hardware network interfaces/antennas, switches, actuators, clock circuits, etc. The described methods and associated actions may also be performed in various orders in addition to the order described in this application, in parallel, and/or simultaneously. Processors of the logic subsystem may be single core or multicore, and the programs executed thereon may be configured for parallel or distributed processing. The logic subsystem may optionally include individual components that are distributed throughout two or more devices, which may be remotely located and/or configured for coordinated processing. One or more aspects of the logic subsystem may be virtualized and executed by remotely accessible networked computing devices configured in a cloud computing configuration.

Aspects of the present disclosure are described herein with reference to flowchart illustrations and/or block diagrams of methods, apparatus, and computer program products according to the embodiments disclosed herein. Insofar as such block diagrams, flowcharts, and/or examples contain one or more functions and/or operations, it will be understood by those of skill in the art that each function and/or operation within such block diagrams, flowcharts, or examples can be implemented, individually and/or collectively, by computer readable instructions using a wide range of hardware, software, firmware, or virtually any combination thereof. The described systems are exemplary in nature, and may include additional elements and/or omit elements. The subject matter of the present disclosure includes all novel and non-obvious combinations and sub-combinations of the various systems and configurations, and other features, functions, and/or properties disclosed.

As used herein, the terms “system” or “module” may include a hardware and/or software system that operates to perform one or more functions. For example, a module or system may include a computer processor, controller, or other logic-based device that performs operations based on instructions stored on a tangible and non-transitory computer readable storage medium, such as a computer memory. Alternatively, a module or system may include a hard-wired device that performs operations based on hard-wired logic of the device. Various modules or units shown in the attached figures may represent the hardware that operates based on software or hardwired instructions, the software that directs hardware to perform the operations, or a combination thereof.

An embodiment provides a restaurant recommendation system comprising: a communication interface for communicating with a user device of a user, a processor, and a storage device storing instructions executable by the processor to: compare user information including dietary restrictions of the user to restaurant information including menu items for a plurality of restaurants, select a restaurant from the plurality of restaurants based on menu items of the restaurant matching the dietary restrictions of the user, and provide a recommendation of the restaurant to the user. In a first example of the system, the user information further comprises a user location, and wherein the instructions are further executable to select the restaurant from the plurality of restaurants in response to a location of the restaurant being less than a threshold distance from the user location. In a second example of the system, optionally including the first example, the instructions are further executable to transmit a notification to the user device alerting the user to the recommendation of the restaurant, the notification including a location and a name of the restaurant. In a third example of the system, optionally including the first and second examples, the notification is transmitted to the user device at a pre-determined time selected by the user. In a fourth example of the system, optionally including the first through third examples, the user information includes data inferred via a machine learning algorithm based on one or more transactions previously conducted by the user. In a fifth example of the system, optionally including the first through fourth examples, the instructions are further executable to select the restaurant from the plurality of restaurants in response to an average review rating of the restaurant exceeding a review rating threshold. In a sixth example of the system, optionally including the first through fifth examples, review data for the restaurant is stored in a reviews database that is stored locally at the restaurant recommendation system or accessible via the communication interface, the reviews database comprising review data for the plurality of restaurants. In a seventh example of the system, optionally including the first through sixth examples, the review data of the reviews database is generated using reviews submitted by users of the restaurant recommendation system and using information from review scraping logic, the review scraping logic including instructions executable by the processor to locate potential sources of menu item information and automatically analyze reviews in the potential sources to identify references to selected menu items. In an eighth example of the system, optionally including the first through seventh examples, providing the recommendation of the restaurant includes: accessing a restaurant profiles database comprising information for a first candidate pool comprising the plurality of restaurants, filtering restaurants from the first candidate pool based on the user information and the restaurant information to generate a second candidate pool comprising a subset of the plurality of restaurants, ranking the restaurants in the second candidate pool, and selecting at least a highest-ranking restaurant from the second candidate pool, wherein the recommendation of the restaurant comprises a recommendation of at least the highest-ranking restaurant. In a ninth example of the system, optionally including the first through eighth examples, ranking the restaurants in the second candidate pool comprises automatically scoring the restaurants in the second candidate pool based on a plurality of scoring factors including one or more of, a distance of each restaurant in the second candidate pool from a user location, a compliance level of menu items of each restaurant in the second candidate pool to the dietary restrictions of the user, and an average review rating of each restaurant in the second candidate pool.

Another embodiment provides a method for recommending a restaurant for a group of users, the method comprising: comparing user information for each user of the group of users to restaurant information for each restaurant of a plurality of restaurants, the user information including dietary restrictions of each user and the restaurant information including menu items for each restaurant, selecting one or more restaurants of the plurality of restaurants that includes menu items meeting the dietary restrictions of each user of the group of users, the one or more restaurants being further selected based on review data for the one or more restaurants, and providing a recommendation of the one or more restaurants to each user of the group of users. In a first example of the method, providing the recommendation of the one or more restaurants includes: accessing a restaurant profiles database comprising information for a first candidate pool comprising the plurality of restaurants, filtering restaurants from the first candidate pool based on the user information for each user of the group of users and the restaurant information to generate a second candidate pool comprising a subset of the plurality of restaurants, ranking the restaurants in the second candidate pool, and selecting at least a highest-ranking restaurant from the second candidate pool, wherein the recommendation of the one or more restaurants comprises a recommendation of at least the highest-ranking restaurant. In a second example of the method, optionally including the first example, ranking the restaurants in the second candidate pool is based on a plurality of scoring factors including one or more of: an estimated travel time between locations of each restaurant in the second candidate pool and a location of the group of users, a number of menu items of each restaurant in the second candidate pool matching the dietary restrictions of the group of users, and an average review rating of each restaurant in the second candidate pool. In a third example of the method, optionally including the first and second examples, ranking the restaurants in the second candidate pool based on the plurality of scoring factors includes: for a restaurant of the restaurants in the second candidate pool: determining one or more of the plurality of scoring factors for the restaurant with respect to each user of the group of users, weighting each of the plurality of scoring factors for each user of the group of users to produce a plurality of weighted factors, and aggregating each of the plurality of weighted factors to produce a weighted average score for the restaurant, wherein a ranking of the restaurant is based on the weighted average score of the restaurant.

An additional embodiment provides a method for recommending restaurants to a user, the method comprising: comparing user information to restaurant information for each restaurant of a plurality of restaurants, the user information including dietary restrictions of the user and the restaurant information including menu items for each restaurant, selecting one or more restaurants of the plurality of restaurants that includes menu items meeting the dietary restrictions of the user, and providing a ranked list of the one or more restaurants to a user device of the user. In a first example of the method, the ranked list comprises a first restaurant of the plurality of restaurants, wherein the first restaurant includes a first number of menu items matching the dietary restrictions of the user, and wherein the ranked list comprises a second restaurant of the plurality of restaurants, wherein the second restaurant includes a second number of menu items matching the dietary restrictions of the user, and wherein the first restaurant is ranked higher than the second restaurant in the ranked list in response to the first number of menu items exceeding the second number of menu items. In a second example of the method, optionally including the first example, the method further comprising: receiving a food order from the user for one or more menu items from a restaurant of the one or more restaurants, and propagating the food order to the restaurant. In a third example of the method, optionally including the first and second examples, the method further comprising: receiving payment information from the user, and propagating the payment information to the restaurant or a payment service associated with the restaurant. In a fourth example of the method, optionally including the first through third examples, the method further comprising: coordinating a delivery, pick up, or dine in experience for the food order, prompting the user to provide a review of the restaurant, and storing the review in a reviews database associated with the restaurant. In a fifth example of the method, optionally including the first through fourth examples, the ranked list comprises a first restaurant of the plurality of restaurants and a second restaurant of the plurality of restaurants, wherein a first ranking of the first restaurant is higher than a second ranking of the second restaurant in response to a first estimated travel time between the first restaurant and a user location being less than a second estimated travel time between the second restaurant and the user location. Each of the example methods may be performed alone or in combination, in any order, using machine readable storage media having machine executable instructions stored thereon that, when executed, cause one or more processors to perform the described methods.

As used in this application, an element or step recited in the singular and proceeded with the word “a” or “an” should be understood as not excluding plural of said elements or steps, unless such exclusion is stated. Furthermore, references to “one embodiment” or “one example” of the present disclosure are not intended to be interpreted as excluding the existence of additional embodiments that also incorporate the recited features. The terms “first,” “second,” and “third,” etc. are used merely as labels, and are not intended to impose numerical requirements or a particular positional order on their objects. The following claims particularly point out subject matter from the above disclosure that is regarded as novel and non-obvious.

1. A restaurant recommendation system comprising:
a communication interface for communicating with a user device of a user;
a processor; and
a storage device storing instructions executable by the processor to:
compare user information including dietary restrictions of the user to restaurant information including menu items for a plurality of restaurants;
select a restaurant from the plurality of restaurants based on menu items of the restaurant matching the dietary restrictions of the user; and
provide a recommendation of the restaurant to the user.

2. The restaurant recommendation system of claim 1, wherein the user information further comprises a user location, and wherein the instructions are further executable to select the restaurant from the plurality of restaurants in response to a location of the restaurant being less than a threshold distance from the user location.

3. The restaurant recommendation system of claim 1, wherein the instructions are further executable to transmit a notification to the user device alerting the user to the recommendation of the restaurant, the notification including a location and a name of the restaurant.

4. The restaurant recommendation system of claim 3, wherein the notification is transmitted to the user device at a pre-determined time selected by the user.

5. The restaurant recommendation system of claim 1, wherein the user information includes data inferred via a machine learning algorithm based on one or more transactions previously conducted by the user.

6. The restaurant recommendation system of claim 1, wherein the instructions are further executable to select the restaurant from the plurality of restaurants in response to an average review rating of the restaurant exceeding a review rating threshold.

7. The restaurant recommendation system of claim 6, wherein review data for the restaurant is stored in a reviews database that is stored locally at the restaurant recommendation system or accessible via the communication interface, the reviews database comprising review data for the plurality of restaurants.

8. The restaurant recommendation system of claim 7, wherein the review data of the reviews database is generated using reviews submitted by users of the restaurant recommendation system and using information from review scraping logic, the review scraping logic including instructions executable by the processor to locate potential sources of menu item information and automatically analyze reviews in the potential sources to identify references to selected menu items.

9. The restaurant recommendation system of claim 1, wherein providing the recommendation of the restaurant includes:
accessing a restaurant profiles database comprising information for a first candidate pool comprising the plurality of restaurants;
filtering restaurants from the first candidate pool based on the user information and the restaurant information to generate a second candidate pool comprising a subset of the plurality of restaurants;
ranking the restaurants in the second candidate pool; and
selecting at least a highest-ranking restaurant from the second candidate pool, wherein the recommendation of the restaurant comprises a recommendation of at least the highest-ranking restaurant.

10. The restaurant recommendation system of claim 9, wherein ranking the restaurants in the second candidate pool comprises automatically scoring the restaurants in the second candidate pool based on a plurality of scoring factors including one or more of;
a distance of each restaurant in the second candidate pool from a user location;
a compliance level of menu items of each restaurant in the second candidate pool to the dietary restrictions of the user; and
an average review rating of each restaurant in the second candidate pool.

11. A method for recommending a restaurant for a group of users, the method comprising:
comparing user information for each user of the group of users to restaurant information for each restaurant of a plurality of restaurants, the user information including dietary restrictions of each user and the restaurant information including menu items for each restaurant;
selecting one or more restaurants of the plurality of restaurants that includes menu items meeting the dietary restrictions of each user of the group of users, the one or more restaurants being further selected based on review data for the one or more restaurants; and
providing a recommendation of the one or more restaurants to each user of the group of users.

12. The method of claim 11, wherein providing the recommendation of the one or more restaurants includes:
accessing a restaurant profiles database comprising information for a first candidate pool comprising the plurality of restaurants;
filtering restaurants from the first candidate pool based on the user information for each user of the group of users and the restaurant information to generate a second candidate pool comprising a subset of the plurality of restaurants;
ranking the restaurants in the second candidate pool; and
selecting at least a highest-ranking restaurant from the second candidate pool, wherein the recommendation of the one or more restaurants comprises a recommendation of at least the highest-ranking restaurant.

13. The method of claim 12, wherein ranking the restaurants in the second candidate pool is based on a plurality of scoring factors including one or more of:
an estimated travel time between locations of each restaurant in the second candidate pool and a location of the group of users;
a number of menu items of each restaurant in the second candidate pool matching the dietary restrictions of the group of users; and
an average review rating of each restaurant in the second candidate pool.

14. The method of claim 13, wherein ranking the restaurants in the second candidate pool based on the plurality of scoring factors includes:
for a restaurant of the restaurants in the second candidate pool:
determining one or more of the plurality of scoring factors for the restaurant with respect to each user of the group of users;
weighting each of the plurality of scoring factors for each user of the group of users to produce a plurality of weighted factors; and
aggregating each of the plurality of weighted factors to produce a weighted average score for the restaurant, wherein a ranking of the restaurant is based on the weighted average score of the restaurant.

15. A method for recommending restaurants to a user, the method comprising:
comparing user information to restaurant information for each restaurant of a plurality of restaurants, the user information including dietary restrictions of the user and the restaurant information including menu items for each restaurant;
selecting one or more restaurants of the plurality of restaurants that includes menu items meeting the dietary restrictions of the user; and
providing a ranked list of the one or more restaurants to a user device of the user.

16. The method of claim 15, wherein the ranked list comprises a first restaurant of the plurality of restaurants, wherein the first restaurant includes a first number of menu items matching the dietary restrictions of the user, and wherein the ranked list comprises a second restaurant of the plurality of restaurants, wherein the second restaurant includes a second number of menu items matching the dietary restrictions of the user, and wherein the first restaurant is ranked higher than the second restaurant in the ranked list in response to the first number of menu items exceeding the second number of menu items.

17. The method of claim 15, the method further comprising:
receiving a food order from the user for one or more menu items from a restaurant of the one or more restaurants; and
propagating the food order to the restaurant.

18. The method of claim 17, the method further comprising:
receiving payment information from the user; and
propagating the payment information to the restaurant or a payment service associated with the restaurant.

19. The method of claim 18, the method further comprising:
coordinating a delivery, pick up, or dine in experience for the food order;
prompting the user to provide a review of the restaurant; and
storing the review in a reviews database associated with the restaurant.

20. The method of claim 15, wherein the ranked list comprises a first restaurant of the plurality of restaurants and a second restaurant of the plurality of restaurants, wherein a first ranking of the first restaurant is higher than a second ranking of the second restaurant in response to a first estimated travel time between the first restaurant and a user location being less than a second estimated travel time between the second restaurant and the user location.
